(19) United States
(12) Patent Application Publication
(10) Pub. No.: US 2023/0305000 A1
(43) Pub. Date: Sep. 28, 2023

(54) SYSTEM FOR RAPID ASSESSMENT OF PECTIN STRUCTURAL/FUNCTIONAL PROPERTIES

(71) Applicant: The United States of America, as represented by the Secretary of Agriculture, Washington, DC (US)

(72) Inventors: WEI ZHAO, FORT PIERCE, FL (US); RANDALL G. CAMERON, Homosassa, FL (US)

(21) Appl. No.: 17/700,713

(22) Filed: Mar. 22, 2022

(51) Int. Cl. G01N 33/543; G01N 33/558; B01L 3/00; G01N 33/58

(52) U.S. Cl. CPC G01N 33/54391; G01N 33/54366; G01N 33/558; G01N 33/54306; B01L 3/5023; G01N 33/58; B01L 2300/0825; B01L 2300/0832; B01L 2200/16

(57) ABSTRACT

The invention relates to systems and methods for the determination of degree and distribution of pectin homogalacturonan (HG) methyl-esterification. The systems comprise a sheet, at least one labeled anti-pectin detection antibody, and a Template Guide. The sheet may comprise an absorbent portion and a membrane portion; an absorbent portion, a membrane portion, and a conjugate portion; or an absorbent portion, a membrane portion, a conjugate portion, and a sample portion. The membrane portion comprises a Control Zone with at least one immobilized unlabeled capture reagent and a Test Zone with at least one immobilized unlabeled anti-pectin capture antibody. When the sheet comprises a conjugate portion, the at least one labeled anti-pectin detection antibody is on the conjugate portion. The system may optionally comprise a container with the at least one labeled anti-pectin detection antibody.

### FIELD OF THE INVENTION

The invention relates to multiplex immunochromatographic systems for the rapid assessment of pectin structural/functional properties.

### BACKGROUND OF THE INVENTION

As a major component of the cell walls in all flowering plant genera, except for the grasses of the Poales, pectin is in every fruit and vegetable consumed by humans. Commercially extracted pectin is widely used as a functional food ingredient in innumerable food products. Pectin is a complex heteropolysaccharide consisting of several domains, predominantly a homogalacturonan (HG) domain and a rhamnogalacturonan I (RG I) domain. The HG domain is a linear homopolymer composed of α-(1-4)-linked D-galacturonic acid (GalA) residues in which some of the C-6 carboxyl groups are methyl-esterified. In nature, HG polysaccharides are synthesized in a highly (around 80%) methyl-esterified form. The degree of methyl-esterification (DM) is decreased to a varying degree during pectin extraction by enzymatic and/or chemical conversion reactions. RG I is a branched polymer with a backbone of a repeating dimer of rhamnose and GalA residues. Galactans, arabinans, or arabinogalactans may be attached to the rhamnose residues as side chains. These branches are often hydrolyzed during pectin extraction procedures and are consequently not usually present in significant amounts in commercially available pectin products.

The global market for pectin surpassed one billion USD per year in 2016 and is expected to reach over two billion USD per year by 2025. The major applications of pectin in food products rely on its gelling and stabilizing properties (functionalities), which are determined primarily by structural features (the degree and pattern of methyl-esterification) of the HG domain. For high-DM pectin (HM; DM>50%), a gel can be formed by the cross-linking of HGs by hydrogen bridges and hydrophobic forces between methoxyl and hydroxyl groups, both are promoted by high sugar concentration and low pH; while for low-DM pectin (LM; DM<50%), a gel is formed by calcium cross-linking between blocks of free contiguous carboxyl groups on HG. In the food industry, HM pectins are used as a gelling agent or stabilizer in sweetened fruit products, confections, beverages, and acidified milk drinks, whereas LM pectins are used in low-calorie foods as they do not require sugars for gelation. The pattern of methyl-esterification (distribution of charge) also influences pectin's functionalities. Pectins with an ordered, block-wise distribution of non-esterified carboxylic acid units have higher gelling quality than those with random distribution. Because a sequence of non-esterified carboxylic acid units is more calcium sensitive (for a LM pectin) than a few consecutive units in gel formation, an HM pectin with long un-esterified blocks is more effective in preventing casein sedimentation in acidified milk drinks.

A number of monoclonal antibodies (mAbs) recognizing pectin structural epitopes are commercially available. They were developed initially for probing pectin structural domains in the context of intact plant cell wall architecture. Among these antibodies, those that bind to HG domains, such as LM19 and LM20, and JIM5 and JIM7 are of particular relevance for the food industry, and their potential for assessment of pectin's structural/functional properties have been explored (Christians, S. et al., 2011, “Anti-homogalacturonan antibodies: A way to explore the effect of processing on pectin in fruits and vegetables?” Food Res. Int. 44: 225-234). However, the used immuno-methods so far (ELISA and Immuno-dot assay) are still laborious and operator-dependent. Most importantly, these methods only distinguish either totally un-esterified or fully esterified (DM>90) pectin, and do not discern the DM, nor the pattern of esterification of partially esterified pectins.

An immunochromatographic assay (ICA) or lateral flow assay is a combination of chromatography and an immunoassay, usually configured in a sandwich or competitive format, suitable for large or small analytes, respectively. Sandwich format ICA involves binding of an analyte molecule to a pair of primary antibodies, while the competitive format ICA involves the competitive binding of analyte molecules to a primary antibody. Both assay formats include immunoreagents immobilized on a carrier and fluid flow through that carrier. This approach allows for adjustable and rapid formation of immune complexes, the use of special zone (Test Zone) to concentrate and to detect target complexes, and the removal of unreacted compounds with the flow from the binding zone. Thus, the technology exhibits significant advantages, such as rapidity and low operational cost (one-step analysis, user-friendly, simple instrumentation), better specificity and higher sensitivity, long term stability under different sets of environmental conditions, and portability. Mass-produced ICA test strips have been very successful in medical diagnostics for point of care or field-based applications, and they are widely used to detect a variety of pathogens and biomarkers in clinical samples.

Pectin's commercial functionality is currently defined by lengthy, laborious, and operator dependent tests commonly known as the SAG, SAM, and YOG tests. Thus, there is an urgent need for a rapid and easy-to-use method to assess pectin's structural/functional properties in many different milieus, including both raw and processed forms, ingredient mixtures, or within formulated foods and feeds.

### SUMMARY OF THE INVENTION

Provided herein are systems or kits for the determination of the degree and distribution of pectin homogalacturonan (HG) methyl-esterification, and methods of using such systems for evaluating the degree and distribution of pectin homogalacturonan methyl-esterification.

In an embodiment, the invention relates to a system for determining HG methyl-esterification. The system comprises a sheet comprising a Control Zone with at least one immobilized unlabeled capture reagent and a Test Zone with at least one immobilized unlabeled anti-pectin capture antibody; at least one labeled anti-pectin detection antibody; and a Template Guide. Where the sheet optionally comprises a conjugate portion or a sample portion and a conjugate portion, and optionally comprises a container. In some embodiments the at least one labeled anti-pectin detection antibody is on the conjugate portion. In some embodiments of the invention the at least one labeled anti-pectin detection antibody is in the container.

In an embodiment, the invention relates to a method for determining pectin's structural/functional properties using a system of the invention. The method comprises contacting at least one labeled anti-pectin detection antibody with a pectin sample to form a pectin/labeled anti-pectin antibody complex; allowing the pectin/labeled antibody complex to flow through the sheet of the system to reach the at least one immobilized unlabeled capture reagent at the Control Zone and the at least one immobilized unlabeled anti-pectin capture antibody at the Test Zone, and to form a color pattern; and comparing the color pattern on the sheet with a Template Guide color pattern. In some embodiments of the invention the at least one labeled anti-pectin detection antibody is in a container, and the pectin sample is added to the container. In some embodiments of the invention the sheet comprises a conjugate portion with the at least one labeled anti-pectin detection antibody, and the pectin sample is added to the conjugate portion. In some embodiments of the invention the sheet comprises a sample portion, and the pectin sample is added to the sample portion.

In some embodiments of the invention, the at least one labeled anti-pectin detection antibody is in a container. In some embodiments of the invention, the at least one labeled anti-pectin detection antibody is on a conjugate pad. In some embodiments of the invention, the at least one unlabeled capture reagent in the system for the detection of degree and distribution of pectin HG methyl-esterification is at least one of an antibody, a protein, an aptamer, or a combination thereof. In some embodiments of the invention the at least one unlabeled anti-pectin capture antibody is a pectin HG-specific antibody. In some embodiments of the invention the at least one labeled anti-pectin detection antibody is a pectin HG-specific antibody.

In some embodiments of the invention, the sheet for assessing pectin's structural/functional properties comprises a membrane portion and an absorbent portion, or comprises a conjugate portion, a membrane portion, and an absorbent portion, or comprises a sample portion, a conjugate portion, a membrane portion, and an absorbent portion. In some embodiments of the invention the immobilized at least one unlabeled capture reagent and the at least one unlabeled anti-pectin capture antibody are in the membrane portion of the sheet.

In some embodiments of the invention, the membrane portion of the sheet comprises two unlabeled capture reagents and three unlabeled anti-pectin capture antibodies. In some embodiments of the invention, the system comprises two anti-pectin detection antibodies labeled with two different labels.

### BRIEF DESCRIPTION OF THE DRAWINGS

FIG. 1 depicts a schematic diagram of the components of an illustrative example of a system for the detection of degree and distribution of pectin HG methyl-esterification taught herein. The system 120 comprises at least one labeled anti-pectin detection antibody 122 which is illustrated in an optional container 124, and a sheet 126 which comprises an absorbent portion 128 and a membrane portion 130. The membrane portion 130 comprises a Control Zone 132 with at least one immobilized unlabeled capture reagent and a Test Zone 134 with at least one immobilized unlabeled capture antibody.

FIG. 2 depicts a schematic diagram of the components of an illustrative example of a system for the detection of degree and distribution of pectin HG methyl-esterification taught herein. The system comprises a sheet 226 comprising an absorbent portion 228, a membrane portion 230, and a conjugate portion 236. The membrane portion 230 comprises a Control Zone 232 with at least one immobilized unlabeled capture reagent and a Test Zone 234 with at least one immobilized unlabeled capture antibody. The conjugate portion 236 comprises at least one labeled anti-pectin detection antibody 222.

FIG. 3 depicts a schematic diagram of the components of an illustrative example of a system for the detection of degree and distribution of pectin HG methyl-esterification taught herein. The system comprises a sheet 326 comprising an absorbent portion 328, a membrane portion 330, a conjugate portion 336, and a sample portion 338. The membrane portion 330 comprises a Control Zone 332 with at least one immobilized unlabeled capture reagent and a Test Zone 334 with at least one immobilized unlabeled capture antibody. The conjugate portion 336 comprises at least one labeled anti-pectin detection antibody 322.

FIG. 4 depicts a schematic diagram of the steps performed when using a system with the components in the illustrative example of FIG. 1 to detect the degree and distribution of pectin HG methyl-esterification in a pectin-containing sample. A pectin-containing sample 440 is contacted with the at least one labeled anti-pectin detection antibody 422, in a container 424, which is optionally supplied with the system. Pectin in the sample 440 binds to the at least one labeled anti-pectin detection antibody 422 forming a pectin/antibody complex 442. The membrane portion 430 of the sheet is put in contact with the pectin/antibody complex 442, and fluid allowed to flow through the membrane portion 430 by capillary action as indicated by the arrow, for a sufficient amount of time for the pectin/antibody complex 442 to reach the Test Zone 434 and the Control Zone 432 to create a Color Pattern, as illustrated.

FIG. 5 depicts photographs of nitrocellulose (NC) membrane strips having immobilized anti-IgM secondary antibody in the Control Zone and immobilized unlabeled LM19 antibody in the Test Zone dipped into vials containing labeled LM19 detection antibody and a dilution of un-esterified pectin. Color at the Control Zone indicates binding of the labeled LM19 detection antibody to the anti-IgM secondary antibody, color at the Test Zone indicates binding of the pectin/LM19 antibody complex to the immobilized unlabeled LM19 antibody. The pectin concentration used for each strip is indicated below the strips in μg/mL. The location of the Control Zone and the Test Zone on the strips is indicated, and an arrow indicates the capillary flow direction.

FIG. 6 depicts a graph of a standard curve based on the relative color intensity obtained on the Test Zone in the NC strips shown in FIG. 6. The Y axis shows the color intensity value quantified using ImageJ software. The X axis shows the pectin concentration in μg/mL. y=7.67921n(x)+38.672. R²=0.9901. Data shown in the graph represents Mean±SEM of 3 independent experiments.

FIG. 7A to FIG. 7D depict images of NC membranes having an immobilized capture reagent in the Control Zone and an immobilized unlabeled antibody in the Test Zone, dipped into vials containing model pectins with a known pattern and degree of methyl-esterification and using different labeled anti-pectin detection antibodies. FIG. 7A shows photographs of NC membranes with an immobilized anti-IgM antibody on the Control Zone and an immobilized LM19 antibody on the Test Zone that were dipped into vials containing model pectins and labeled LM19 detection antibody. FIG. 7B shows photographs of NC membranes with an immobilized anti-IgM antibody on the Control Zone and an immobilized LM20 antibody on the Test Zone that were dipped into vials containing model pectins and labeled LM20 detection antibody. FIG. 7C shows photographs of NC membranes with an immobilized anti-IgG antibody on the Control Zone and an immobilized JIM5 antibody on the Test Zone that were dipped into vials containing model pectins and labeled JIM5 detection antibody. FIG. 7D shows photographs of NC membranes with an immobilized anti-IgA antibody on the Control Zone and an immobilized JIM7 antibody on the Test Zone that were dipped into vials containing model pectins and labeled JIM7 detection antibody. The model pectins and their structural properties are indicated at the top of the figure.

FIG. 8A to FIG. 8D depict graphs of the quantified visual results shown in FIG. 7A to FIG. 7D. FIG. 8A shows the results obtained when using LM19 antibody. FIG. 8B shows the results obtained when using LM20 antibody. FIG. 8C shows the results obtained when using JIM5 antibody. FIG. 8D shows the results obtained when using JIM7 antibody. The Y axis shows the color intensity. The X axis indicates the model pectins used and their structural properties.

FIG. 9 depicts a schematic diagram of an illustrative membrane portion set up for use with a single labeled anti-pectin detection antibody in a system of the invention. The illustrative membrane portion contains unlabeled and immobilized at a Control Zone an anti-IgA antibody, and either an anti-IgM antibody or an anti-IgG antibody. The illustrative membrane portion contains a Test Zone with immobilized unlabeled capture antibodies JIM5, JIM7, LM19, and LM20. The capillary flow direction is indicated by an arrow.

FIG. 10A to FIG. 10D depict images of membrane portions as depicted in FIG. 9 used to test model pectins with a known pattern and degree of methyl-esterification using different labeled anti-pectin detection antibodies. FIG. 10A shows photographs of membrane portions tested using labeled LM19 detection antibody. FIG. 10B shows photographs of membrane portions tested using labeled LM20 detection antibody. FIG. 10C shows photographs of membrane portions tested using labeled JIM5 detection antibody. FIG. 10D shows photographs of membrane portions tested using labeled JIM7 detection antibody. The model pectins and their structural properties are indicated at the top of the figure.

FIG. 11 depicts a schematic diagram of an illustrative membrane portion designed to be used for assessment of pectin structural/functional properties using two anti-pectin detection antibodies with different labels in a system of the invention. The illustrative membrane portion contains a Control Zone with an immobilized anti-IgM secondary antibody and an immobilized anti-IgA secondary antibody; and a Test Zone with immobilized unlabeled JIM7, LM19, and LM20 capture antibodies.

FIG. 12 depicts images of membrane portions of a system of the invention as depicted in FIG. 11 used to analyze model pectins with a known pattern and degree of methyl-esterification with a JIM7 anti-pectin antibody labeled with 80 nm spherical GNPs and an LM20 anti-pectin antibody labeled with 80 nm star-shaped GNPs. The type of methyl-esterification distribution, the degree of methoxyl present (LM or HM), and the name of the model pectin tested are indicated above the membrane images.

FIG. 13 depicts images of membrane portions of a system of the invention as depicted in FIG. 11 used to analyze commercial pectins with a known pattern and degree of methyl-esterification with a JIM7 anti-pectin antibody labeled with 80 nm spherical GNPs and an LM20 anti-pectin antibody labeled with 80 nm star-shaped GNPs. The different commercial pectins tested and the degree of methoxyl present (either low (LM) or high (HM)) are indicated above the membrane portions. Commercial-1 is GENU pectin type LM 12 CG-Z; Commercial-2 is GENU Explorer pectin 60 CS; Commercial-3 is GENU pectin type YM-115-L; Commercial-4 is SLENDID specialty pectin type 200.

FIG. 14 depicts a Template Guide for calling the results when a system of the invention with a membrane portion as diagrammed in FIG. 11 and using a JIM7 anti-pectin antibody labeled with 80 nm spherical GNPs and a LM20 anti-pectin antibody labeled with 80 nm star-shaped GNPs as detection antibodies. A filled gray dot indicates a blue color; a dot with vertical stripes indicates a red color; a dot with upward diagonal stripes indicates a purple color; a dot with downward diagonal stripes indicates a dark purple color.

### DETAILED DESCRIPTION

The present invention relates to multiplex immunochromatographic systems for the rapid assessment of pectin structural/functional properties.

Pectin is a multi-faceted functional ingredient widely used in the food, pharmaceutical, and personal care industries. Based on the pectin functional properties it may be used for different purposes, which are primarily determined by the structural properties (degree and pattern of methyl-esterification) of its HG backbone. Currently, pectin's commercial functionality is defined by lengthy, laborious, and operator-dependent tests. The inventors have developed a novel, rapid, easy to use multiplex immunochromatographic assay (ICA) test strip to detect and assess pectin structural properties that are related to its functionality. The test strip may include three different capture antibodies immobilized on a test strip and two detection antibodies labeled with different colors of gold nanoparticles that may be lyophilized in a small vial. A user needs only to drop pectin sample into the small vial, insert the test strip into the vial, then wait five (5) minutes to obtain results. Compared to a conventional immuno-method, the method taught herein is not only much faster, but it is also more sensitive, with a detection limit of 0.02 ppm of pectin. Most importantly, the ICA test strip taught here is able to discern the degree and pattern of methyl-esterification of pectin. Capability of the test strip has been demonstrated by a series of experimental pectins and several commercial pectins. The technology can be easily scaled up to high-volume manufacturing. The convenience of such an ICA test strip would benefit the entire spectrum of the pectin utilization chain, from producers and ingredient suppliers to food formulators and those in the medical and personal care industries.

A series of pectins with different distribution patterns may be prepared by alkaline de-esterification; by using a fungal pectin methyl-esterase; or by using a plant methyl-esterase. In the instant disclosure, model pectins with a different degree of methyl-esterification were used. The “0” indicates polygalacturonic acid with no methyl esters, the “94” indicates that the parent pectin from which all the other samples were produced, has a 94% degree of methyl-esterification. Pectin samples with a random distribution produced by alkaline de-esterification are referred to as “S” series pectins. In S28 there is a 28% degree of randomly distributed methyl-esterification. In S53 there is a 53% degree of randomly distributed methyl-esterification. In S72 there is a 72% degree of randomly distributed methyl-esterification. In S81 there is an 81% degree of randomly distributed methyl-esterification. Pectin samples with a semi-random distribution produced using a fungal pectin methyl-esterase are referred as “F” series pectins. In F30 there is a 30% degree of semi-randomly distributed methyl-esterification. In F50 there is a 50% degree of semi-randomly distributed methyl-esterification. In F60 there is a 60% degree of semi-randomly distributed methyl-esterification. In F70 there is a 70% degree of semi-randomly distributed methyl-esterification. In F80 there is an 80% degree of semi-randomly distributed methyl-esterification. Pectin samples with block-wise distribution produced using a plant pectin methyl-esterase are referred to as “P” series pectins. In P30 there is a 30% degree of block-wisely distributed methyl-esterification. In P47 there is a 47% degree of block-wisely distributed methyl-esterification. In P70 there is a 70% degree of block-wisely distributed methyl-esterification.

In the instant disclosure, commercially-available pectins tested were GENU pectin type LM 12 CG-Z, DM 35% (referred to as Commercial-1); GENU Explorer pectin 60 CS, DM 38% (referred to as Commercial-2); GENU pectin type YM-115-L, DM 72% (referred to as Commercial-3); and SLENDID specialty pectin type 200, labeled as HM pectin but its DM was not provided (referred to as Commercial-4).

An immunochromatographic assay (ICA) or lateral flow test is a device simple-to-use to confirm the presence or absence of a target substance. Different industry sectors and different countries use varying terminology to describe a lateral flow test. Common names include lateral flow test (LFT), lateral flow device (LFD), lateral flow assay (LFA), lateral flow immunoassay (LFIA), lateral flow immunochromatographic assay, dipstick, express test, pen-side test, quick test, rapid test, and test strip. Due to their versatile nature, lateral flow rapid tests are used across a number of industry sectors including medical diagnostics, pharmaceuticals, environmental testing, animal health, food and feed testing, and plant and crop health.

Lateral flow tests run the liquid sample along the surface of a pad with reactive molecules that show a visual positive or negative result. The pads are based on a series of capillary beds such as pieces of porous paper, microstructured polymers, and sintered particles, which have the capacity to spontaneously transport fluid.

A typical lateral flow test strip is composed of four orderly assembled components: a sample pad, a conjugate pad, a nitrocellulose (NC) membrane, and an absorbent pad. There is a small overlap (about 2 mm) between all of these components, allowing the sample to move through the test strip via capillary action.

Current known lateral flow tests operate as either competitive assays or sandwich assays. A sandwich format test involves binding of an analyte molecule to a pair of primary antibodies (labeled detection antibody and unlabeled capture antibody) while a competitive format test involves the competitive binding of analyte molecules to a primary antibody. In a sandwich assay format, the conjugate pad temporarily adsorbs labeled antibody, which can be flushed away by the sample flow. A capture antibody against target analyte is immobilized on the NC membrane. Sample containing the analyte is applied to the sample pad and it migrates to the conjugate pad, where target analyte binds to the detection antibody and forms an analyte-labeled antibody complex. This complex travels to, and flows through, the NC membrane until it is captured by the capture antibody, resulting in visible color there. Buffer or excess reagent wicks to the absorbent pad.

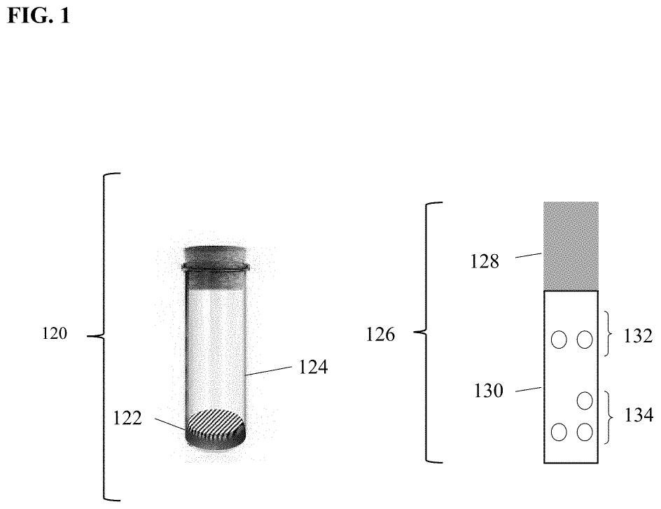

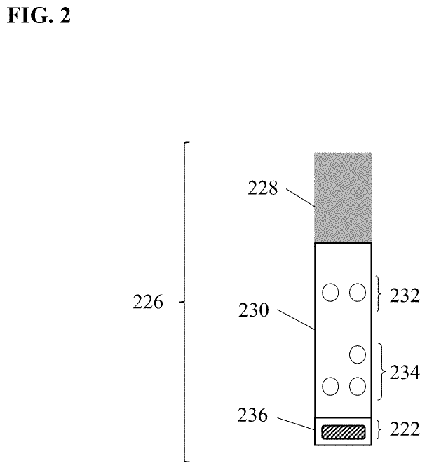

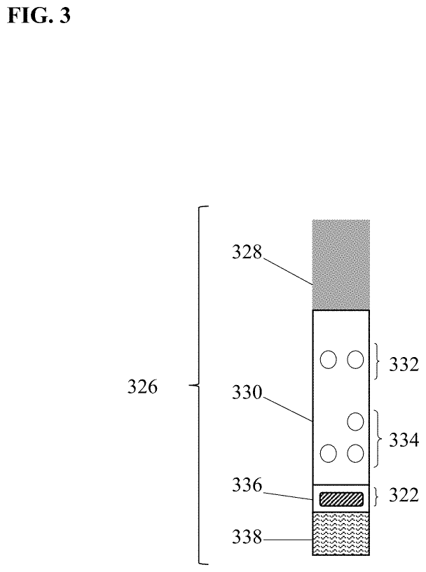

As shown in FIG. 1, a kit or system for an assay of the invention may comprise at least one labeled anti-pectin detection antibody 122 which may be in an optional container 124, and a sheet 126 comprising an absorbent portion 128 and a membrane portion 130. The membrane portion 130 may comprise a Control Zone 132 containing at least one immobilized unlabeled capture reagent that may capture the at least one labeled anti-pectin detection antibody, and a Test Zone 134 containing at least one immobilized unlabeled anti-pectin capture antibody that may specifically capture a pectin in a sample. As shown in FIG. 2, a kit or system for an assay of the invention may comprise a sheet 226 comprising an absorbent portion 228, a membrane portion 230, and a conjugate portion 236. The membrane portion 230 may comprise a Control Zone 232 containing at least one immobilized unlabeled capture reagent that may capture the at least one labeled anti-pectin detection antibody, and a Test Zone 234 containing at least one immobilized unlabeled anti-pectin capture antibody that may specifically capture a pectin in a sample. The conjugate portion 236 may comprise at least one labeled anti-pectin detection antibody 222. As shown in FIG. 3, a kit or system for an assay of the invention may comprise a sheet 326 comprising an absorbent portion 328, a membrane portion 330, a conjugate portion 336, and a sample portion 338. The membrane portion 330 may comprise a Control Zone 332 containing at least one immobilized unlabeled capture reagent that may capture the at least one labeled anti-pectin detection antibody, and a Test Zone 334 containing at least one immobilized unlabeled anti-pectin capture antibody that may specifically capture a pectin in a sample. The conjugate portion 336 may comprise at least one labeled anti-pectin detection antibody 322.

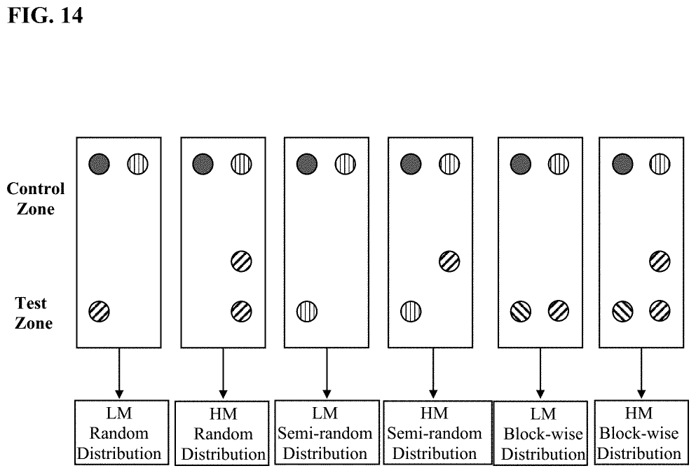

The kit may also comprise a Template Guide, an example of which is shown in FIG. 14.

Provided herein are systems, methods, and kits relating to the rapid assessment of pectin structural/functional properties. Exemplary protocols for the systems, assays, and methods described herein can be found in the Examples. The general premise of the assays and variations thereof are described briefly below.

The systems of the invention comprise a sheet, at least one labeled anti-pectin detection antibody, and a Template Guide. The at least one labeled anti-pectin detection antibody may optionally be in a container. The at least one labeled anti-pectin detection antibody may be lyophilized and may be in the presence and/or absence of other substances. Substances present with the at least one labeled anti-pectin detection antibody may be a buffer, a surfactant, a blocking agent, or a mixture thereof. Examples of substances that may be present with the at least one labeled anti-pectin detection antibody are phosphate buffered saline, sucrose, glucose, tris(hydroxymethyl)aminomethane (Tris), ethylenediaminetetraacetic acid (EDTA), 4-(2-hydroxyethyl)-1-piperazineethanesulfonic acid (HEPES), sorbitol, trehalose, polysorbate 20, and BSA.

The at least one labeled anti-pectin detection antibody may be labeled with any substance which is visible by itself, or which is capable of producing a signal that is detectable by visual or instrumental means. The selection of a particular label is not critical to the invention, but the label should be capable of generating a detectable signal by itself, or be instrumentally detectable, or be detectable in conjunction with one or more additional signal-producing components such as an enzyme and or a substrate signal-producing enzyme. When using two labeled anti-pectin detection antibodies, each antibody should be labeled with a different substance, which preferable

As is known in the art the choice of the type of label involves consideration of the analyte to be detected and the desired means of detection. Detection of the detectable label will depend on the chosen moiety or reagent, and can be done by any of the methods known in the art, for example, visual inspection, ultraviolet and visible spectrophotometry, fluorimetry, radioactivity counting, or the like. Various labels known in the art and suitable for use in the methods of the invention include labels which are visible themselves or which produce signals through either chemical or physical means. Such labels can include enzymes and substrates, chromogens, catalysts, fluorescent compounds, chemiluminescent compounds, molecular beacons, carbon black, polystyrene beads, and radioactive labels. Other suitable labels known in the art include particulate labels such as colloidal metallic particles such as colloidal gold, colloidal non-metallic particles such as selenium or tellurium, dyed or colored particles such as a dyed plastic or a stained microorganism, organic polymer latex particles and liposomes, colored beads, polymer microcapsules, sacs, erythrocytes, erythrocyte ghosts, or other vesicles containing directly visible substances, and the like. Use of a visually detectable label allows for direct visual or instrumental reading of the presence or amount of an analyte in the sample without the need for additional signal producing components at the test zone. Methods to attach labels to antibodies are known in the art. See e.g., Hermanson, G. T., 2013, Bioconjugate Techniques, Third Ed., Academic Press, Elsevier Inc., London, UK. In an embodiment, when gold particles are used as detectable labels, the results of the assay are obtained and analyzed by visual inspection.

The detectable label may be a metal colloidal particle, such as gold microparticles or gold nanoparticles, which can be seen by the human eye as well as read with an instrument. Colloidal particles used as a detectable label have a particle size diameter of between 20 nm and 150 nm. For an instrument-read assay, the concentration of the particles is measured by measuring Optical Density (OD) at a specific wavelength using a spectrophotometer. The OD at the Test Zone is then used as a measure of the amount of labeled antibody bound to pectin and the OD at the Control Zone is used as a measure of unbound labeled antibody.

In the system of the invention the at least one labeled anti-pectin detection antibody specifically detects a pectin structural/functional property. The at least one labeled anti-pectin detection antibody may be at least one of JIM5 antibody, JIM7 antibody, LM19 antibody, or LM20 antibody. In some embodiments of the invention, the labeled anti-pectin detection antibodies in a system of the invention are JIM7 and LM20.

A capture reagent is a component which specifically binds to antibodies of a specific class but not to antibodies of another class. The constant domains of the heavy chains make up the Fc region of an antibody. The Fc region of the antibody determines the antibody class. Therefore, capture reagents for a particular antibody class specifically recognize the Fc regions. The capture reagent may be an antibody, a protein, an aptamer, or other specific affinity reagent that recognizes a class of antibodies. For example, Protein G, as known in the art, binds to all subclasses of human IgG, but not human IgM. Protein A binds to certain subclasses of IgG as well as other classes of antibodies. Antibodies specific for a class of antibody may also be used as capture reagents. For example, an anti-IgM antibody or a fragment thereof may be used to capture IgM; an anti-IgG antibody or a fragment thereof may be used to capture IgG; an anti-IgA antibody or a fragment thereof may be used to capture IgA.

Capture reagents may be immobilized at specified regions on a surface to form Capture Zones. For example, capture reagents may be immobilized on specific regions, areas, zones, or lines on a lateral flow assay test strip. Immobilization of capture reagents onto a Capture Zone of a lateral flow assay strip may be done using techniques known in the art. In an example of the instant disclosure the strip may contain two different Capture Zones, a Control Zone containing at least one capture reagent, and a Test Zone containing at least one unlabeled anti-pectin capture antibody.

In an embodiment of the invention, the sheet of the invention may comprise an absorbent portion and a membrane portion. The absorbent portion may be any material capable of absorbing or draining a fluid by capillary action. Most absorbent portions are made of cellulose fibers. In an embodiment of the invention, the sheet may comprise a sample portion and a conjugate portion. The material used for the membrane portion is not particularly limited, so long as it is a material enabling immunochromatography. Examples of such may be a fiber matrix of cellulose derivative, a filter paper, a glass fiber, a cloth, a cotton, a polyester, an acrylonitrile, or nylon. In some embodiments, the membrane portion in a system of the invention is nitrocellulose.

The sample portion may be made of any material and in any form allowing the passage of liquid and the component to be detected. Specific examples include, but are not limited to, a glass fiber, an acrylic fiber, a hydrophilic polyethylene material, a dry paper, a paper pulp, a fabric, etc.

The sheet in a system of the invention may comprise a membrane portion containing a Control Zone with at least one immobilized capture reagent, and a Test Zone containing at least one immobilized unlabeled anti-pectin capture antibody. The specific location of the Test Zone and the Control Zone on the membrane are not important, as long as the capillary action allows the pectin sample and the at least one labeled anti-pectin detection antibody to travel through the Control Zone and the Test Zone. The direction of capillary flow through the membrane is irrelevant. In some embodiments of the invention the at least one labeled antibody travels by capillary action first through the Test Zone and then through the Control Zone. In some embodiments of the invention, the at least one labeled antibody travels by capillary action first through the Control Zone and then through the Test Zone. In some embodiments of the invention, the strip may be wide enough so that the Control Zone and the Test Zone may be at the same level and the capillary flow reaches both zones at the same time.

The detection limit of the assay system of the invention was determined using NC membranes prepared with an anti-IgM secondary antibody immobilized in the Control Zone and unlabeled LM19 antibody immobilized in the Test Zone. Serial dilutions of totally un-esterified pectin (20 μL) were added into individual vials containing labeled LM19 antibody, then the test strips were separately inserted into the vails. The sample-labeled antibody flowed through the Test Zone and the Control Zone via capillary action. The different pectin dilutions were 10 μg/mL, 5 μg/mL, 2.5 μg/mL, 1.25 μg/mL, 0.63 μg/mL, 0.31 μg/mL, 0.16 μg/mL, 0.08 μg/mL, 0.04 μg/mL, 0.02 μg/mL, 0.01 μg/mL, and 0.005 μg/mL.

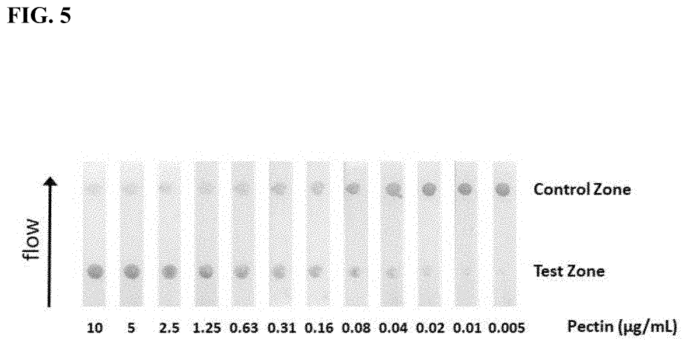

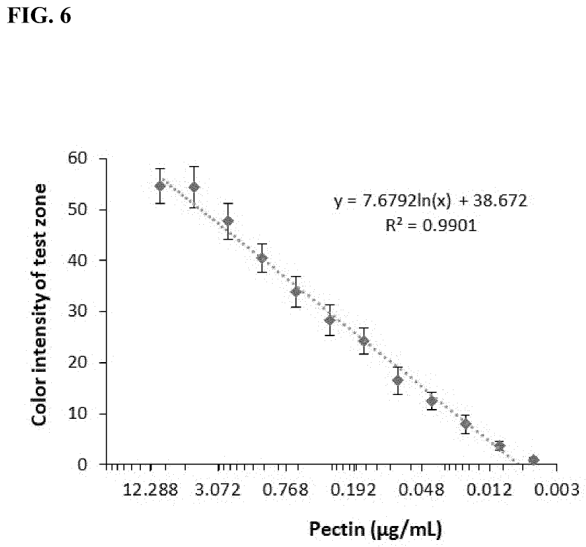

After a pectin sample was contacted with the at least one labeled anti-pectin detection antibody, the pectin samples with the labeled antibody to flowed through the NC sheets, and color appeared at the Control Zone and at the Test Zone. Appearance of color at the Control Zone indicated that the at least one detection antibody had been labeled successfully and that the fluid flowed by capillary action through the NC membrane. Appearance of color at the Test Zone indicated that pectin had bound to the at least one labeled anti-pectin detection antibody and to the immobilized anti-pectin capture antibody, indicating a positive result (as in a sandwich ICA). FIG. 5 shows photographs of NC membranes tested with different pectin dilutions and with labeled LM19 antibody. Not wishing to be bound by theory, it is believed that when using 10 μg/mL pectin the signal is darker at the Test Zone and lighter at the Control Zone, indicating that most of the labeled anti-pectin detection antibody was bound to pectin and captured by capture antibody at the Test Zone, and little of it was left “non-loaded” to reach the Control Zone. When using 0.02 μg/mL pectin the signal is lighter at the Test Zone and darker at the Control Zone, indicating there were very few pectin molecules present in the sample, thus most of the labeled detection antibody was not bound to pectin. The “non-loaded” labeled antibody passed through the Test Zone, and bound to the secondary antibody at the Control Zone. At 0.02 μg/mL or a higher concentration, the signal at the Test Zone was visible; while at 0.01 μg/mL or 0.005 μg/mL, the signal at the Test Zone was barely or not visible by eyes. The results indicate the detection limit of the assay taught herein is about 0.02 μg/mL. Color intensity at the Test Zones of the strips was quantified by ImageJ software, a standard curve was generated based on the color intensity, and is shown in FIG. 6. The data shown in the graph represents Mean±SEM of 3 independent experiments. This graph shows that there was a good correlation (R²=0.99) between the concentration of the pectin and the color intensity in the Test Zone, and the detection limit of the assay taught herein is between 0.01 μg/mL and 0.02 μg/mL. Not wanting to be bound by theory, it is believed that the reason why the method taught herein is so sensitive is because signal from the labeled anti-pectin detection antibody is concentrated and amplified by the binding of the pectin/labeled anti-pectin detection antibody to the unlabeled capture antibody in a small area at the Test Zone. This results in a dramatically increased sensitivity and a much lower detection limit compared to conventional immunoassays. The lower detection limit makes the method taught herein applicable to a wider range of samples including those with low pectin content. The results shown in FIG. 5 and FIG. 6 indicate that the detection limit of the pectin assay system of the invention is about 500 times more sensitive than that of Immuno-dot assay (IDA).

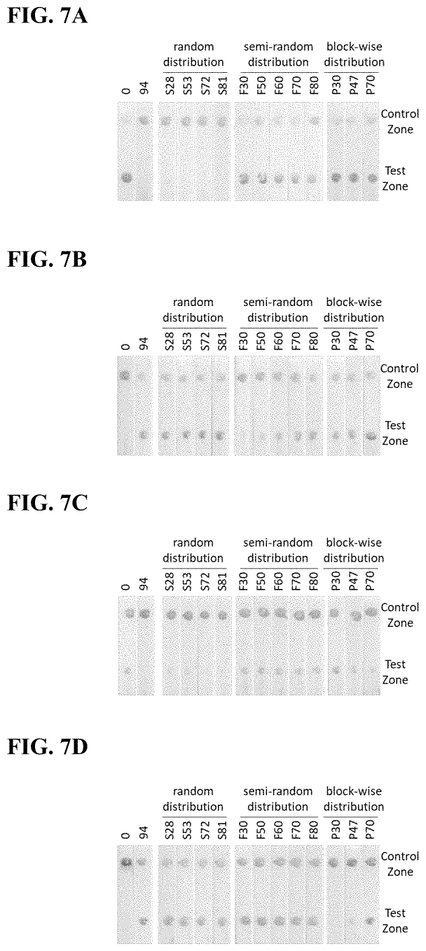

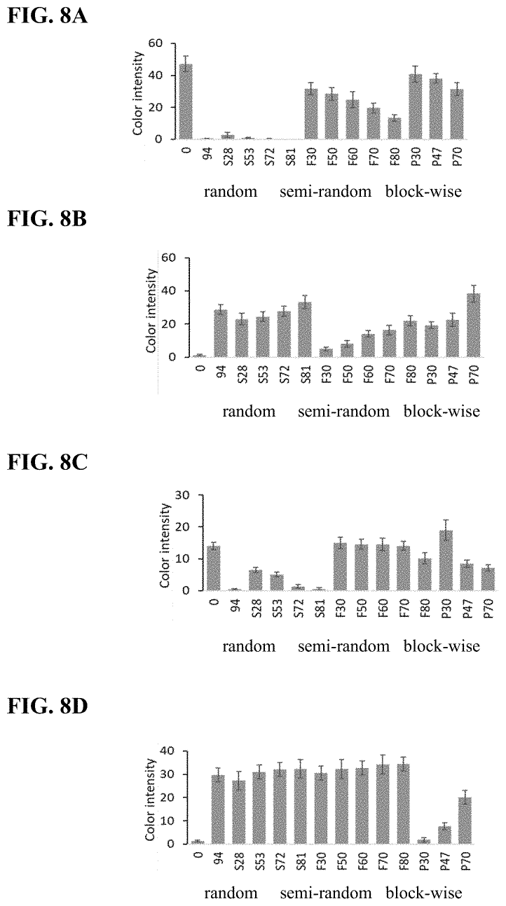

The abilities of antibodies LM19, LM20, JIM5 and JIM7 to discriminate pectin structural properties in ICA were evaluated with a series of model pectins with a known pattern and degree of methyl-esterification. Antibodies LM19, LM20, JIM5 and JIM7 were labeled with GNPs and used as detection antibodies separately; and the unlabeled antibodies were used as capture antibodies separately on the Test Zone of test strips, and their respective secondary antibodies were on the Control Zone. Appearance of a positive signal (color) on the Test Zone was an indication of the formation of a “sandwich” where pectin was captured between an immobilized unlabeled capture antibody and a labeled detection antibody. The results obtained when using LM19 antibody in the tests are shown in FIG. 7A. This figure shows positive results for un-esterified pectin (0), partially de-esterified pectins with a semi-random distribution (F30, F50, F60, F70, and F80), or pectins with an ordered, block-wise distribution (P30, P47, and P70) of methyl-esterification; while showing negative results for fully-esterified pectin (DM>90%) and partially de-esterified pectins with a random distribution of methyl-esterification (S28, S53, S72, and S81). The results obtained when using LM20 antibody in the tests are shown in FIG. 7B. This figure shows positive results for fully or partially esterified pectins, but the signals were weak for those partially esterified pectins with a semi-random distribution of methyl esterification. The LM20 antibody did not generate a positive result for un-esterified (0) pectin. The results obtained when using JIM5 antibody in the tests are shown in FIG. 7C. This figure indicates that the results of JIM5 were similar to that of the LM19, but the signals were much weaker. The results obtained when using JIM7 antibody in the tests are shown in FIG. 7D. This figure shows positive results for fully or partially esterified pectins except the LM (DM<50%) pectin with a block-wise distribution of methyl esterification; and a negative result for un-esterified pectin (0). The color intensity in FIG. 7A to FIG. 7D was quantified using ImageJ software and the results are shown in FIG. 8A to FIG. 8D.

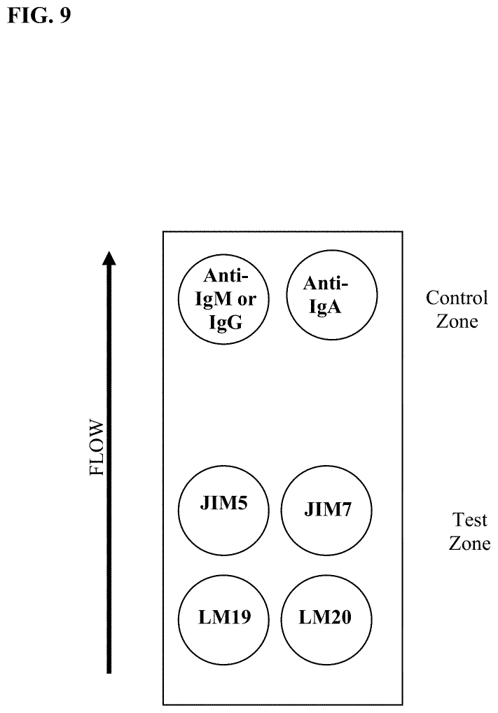

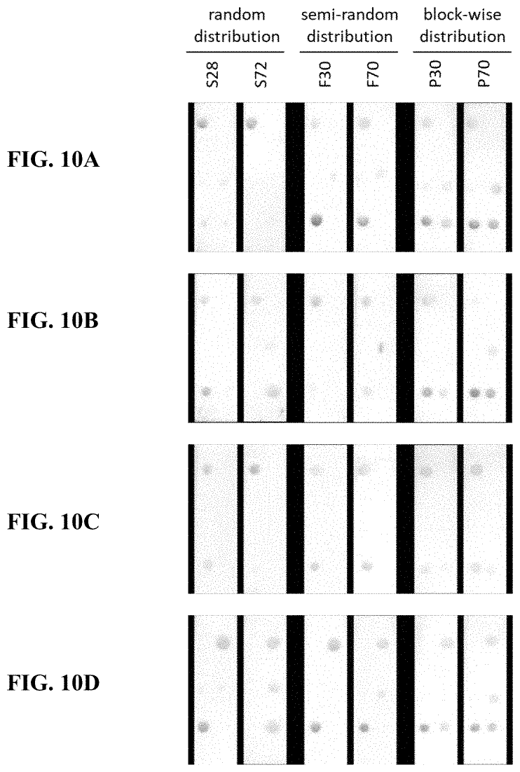

A schematic diagram of an illustrative example of a membrane portion of a system of the invention including two secondary antibodies (anti-IgM or anti-IgG and anti-IgA) in the Control Zone, and four capture antibodies (JIM5, JIM7, LM19, and LM20) immobilized in the Test Zone is depicted in FIG. 9. The system including four capture antibodies as illustrated in FIG. 9 and was evaluated by testing model pectins with a known pattern and degree of methyl-esterification using one labeled detection antibody (LM19, LM20, JIM5, or JIM7). Tested were model pectins S28 and S72 of random distribution, F30 and F70 of semi-random distribution, and P30 and P70 of block-wise distribution. FIG. 10A shows the test results when using GNP-labeled LM19 detection antibody. The results indicate LM19 detection antibody discriminated between the different distributions; but generated very weak signals for pectins with random distribution. FIG. 10B shows the test results when using GNP-labeled LM20 detection antibody. The results indicate LM20 detection antibody discriminated between the different types of pectins by showing a specific signal pattern for each of the pectin types. However, there was no positive signal for the semi-randomly distributed LM pectin (F30). FIG. 10C shows the test results when using GNP-labeled JIM5 detection antibody. The results indicate JIM5 detection antibody did not clearly discriminate between the different pectins. FIG. 10D shows the test results when using GNP-labeled JIM7 detection antibody. The results indicate JIM7 detection antibody discriminated between LM and HM pectins and generated at least one positive signal for all the pectin types; but it could not distinguish between randomly and semi-randomly distributed LMs (S28 and F30).

It is possible that simultaneous use of labeled LM20 and JIM7 detection antibodies in one test will increase the signals at the immobilized JIM7 on the Test Zone for HM pectin samples due to an “additive effect”. In addition, if LM20 and JIM7 are labeled with different colors, it would also be possible to differentiate between randomly and semi-randomly distributed LMs (S28 and F30) by showing different colors on the immobilized LM19 capture antibody. The color of a Gold Nanoparticle (GNP) depends on its size and shape, and 80 nm GNPs work best for pectin samples. The 80 nm spherical GNPs are red, and the 80 nm star-shaped GNPs (Gold NanoUrchins) are blue. Thus, they would be a good set for labeling the two antibodies (LM20 and JIM7). As for the array of capture antibodies, since there is no positive signal on the JIM5 capture antibody site by LM20 or JIM7 detection antibody for all the pectin samples, JIM5 can be excluded.

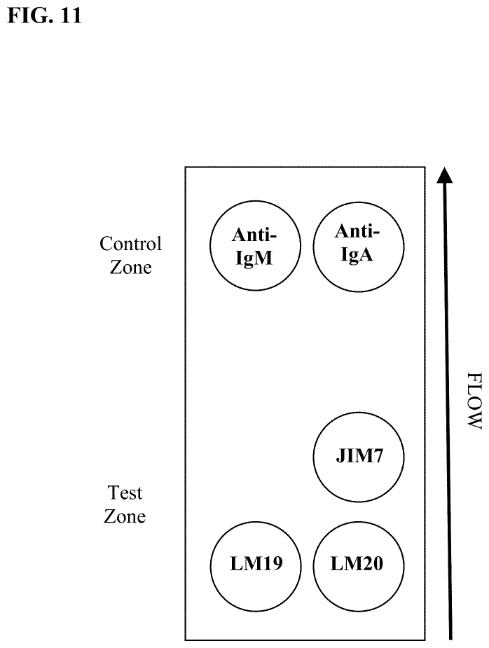

An illustrative multiplex ICA system was developed, which includes a membrane portion with two secondary antibodies (anti-IgM and anti-IgA) at the Control Zone and three capture antibodies (LM19, LM20 and JIM7) at the Test Zone, as schematically diagrammed in FIG. 11, and using two anti-pectin detection antibodies labeled with different colors (80 nm spherical GNP-labeled JIM7 (red) and 80 nm star-shaped GNP-labeled LM20 (blue)). The ability of the system to discern different structural/functional forms of pectin was evaluated by testing model pectins and commercial pectins.

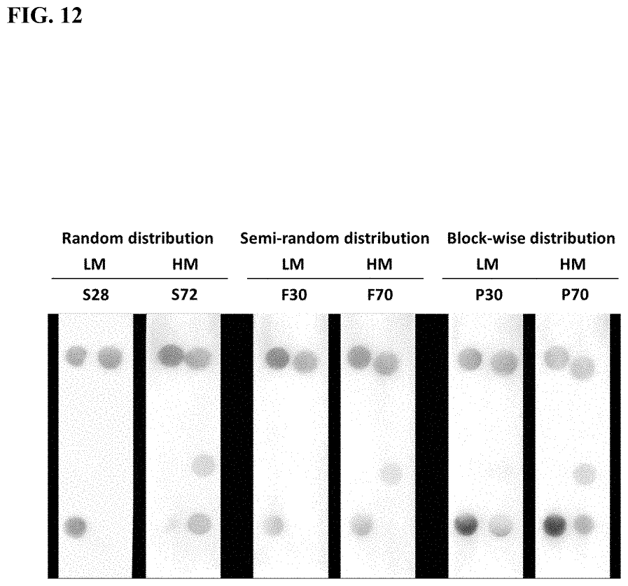

Tested were model pectins with random distribution S28 (LM) and S72 (HM); with semi-random distribution F30 (LM) and F70 (HM); and with block-wise distribution P30 (LM) and P70 (HM). The results are depicted in FIG. 12, which shows that a specific pattern or color of positive signal in the Test Zone was obtained with each of the model pectins tested. These results indicated the developed ICA system is capable of discriminating between different structural types of pectin that are related to pectin's functionality.

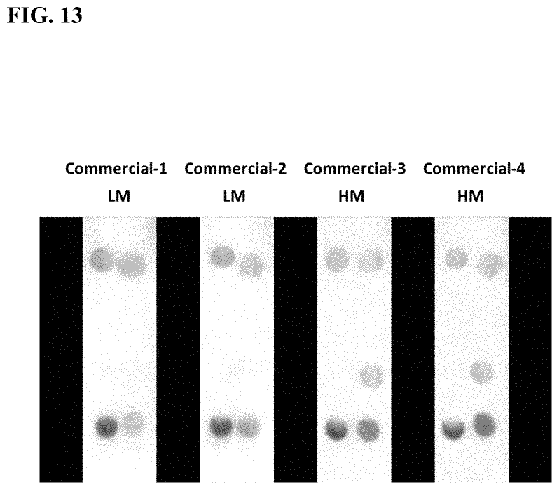

Commercial pectins tested were GENU pectin type LM 12 CG-Z, DM 35% and GENU Explorer pectin 60 CS (both LM pectins), DM 38%; GENU pectin type YM-115-L, DM 72% and SLENDID specialty pectin type 200 (both HMs). SLENDID specialty pectin type 200 pectin is labeled as an HM pectin, but its DM was not provided. These four commercial pectins were tested using a system of the invention with a membrane portion as depicted in FIG. 11, and using a JIM7 antibody labeled with 80 nm spherical GNPs (red) and a LM20 antibody labeled with 80 nm star-shaped GNPs (blue). Photographs of the results obtained are presented in FIG. 13. In this figure the pectins tested and their degree of methoxyl are indicated at the top of the figure. Commercial-1 stands for GENU pectin type LM 12 CG-Z, DM 35%; Commercial-2 stands for GENU Explorer pectin 60 CS, DM 38%; Commercial-3 stands for GENU pectin type YM-115-L, DM 72%; Commercial-4 stands for SLENDID specialty pectin type 200, HM.

The results shown in FIG. 13 indicate that these commercial pectin products have block-wise distribution of methyl-esterification, which is a desirable trait for a gelling agent or a stabilizer to be used in foods. These results also show different patterns of positive signals in the Test Zone for LM pectins and HM pectins. These results were consistent with the information provided by the provider of the commercial pectins. These results indicate that it is possible to assess commercial pectins by using a system of the invention with a membrane portion as depicted in the illustrative example of FIG. 11, using a JIM7 detection antibody labeled with 80 nm spherical GNPs, and a LM20 detection antibody labeled with 80 nm star-shaped GNPs.

Based on the experimental data obtained, a judgment criteria for pectin structural properties tested using a multiplex ICA system of the invention may be summarized in a Template Guide as depicted in FIG. 14. It is possible to determine the degree and distribution of pectin HG methyl-esterification using any one of the systems whose schematic diagram is depicted in any one of FIG. 1, FIG. 2, or FIG. 3 with an anti-IgM and an anti-IgA immobilized at the Control Zone, a JIM7 antibody, an LM19 antibody, and an LM20 antibody immobilized at the Test Zone, and using a JIM7 antibody labeled with 80 nm spherical GNPs and a LM20 antibody labeled with 80 nm star-shaped GNPs (Gold NanoUrchins).

Appearance of one blue dot and one red dot on the Control Zone indicated that the system is functioning properly. The results were judged based on the pattern/color of the dots appearing on the Test Zone. A developed test strip with only one purple dot on the immobilized LM19 at the Test Zone indicated a pectin with LM random distribution. A developed test strip with two purple dots (one on the immobilized JIM7 and one on the immobilized LM20) at the Test Zone indicated a pectin with HM random distribution. A developed test strip with only one red dot on the immobilized LM19 at the Test Zone indicated a pectin with LM semi-random distribution. A developed test strip with one red dot on the immobilized LM19 and one purple dot on the immobilized JIM7 at the Test Zone indicated a pectin with HM semi-random distribution. A developed test strip with one dark purple dot on the immobilized LM19 and a lighter purple dot on the immobilized LM20 at the Test Zone indicated a pectin with LM block-wise distribution. A developed test strip with one dark purple dot on the immobilized LM19 and two lighter purple dots, one on the immobilized LM20 and one on the immobilized JIM7 at the Test Zone indicated a pectin with HM block-wise distribution.

Examples of possible arrangements of the secondary antibodies and unlabeled capture antibodies on the membrane are shown in the figures. One of skill in the art understands that the exact arrangement of the capture reagents and unlabeled capture antibodies on the membrane is not relevant as long as the position of the antibodies on the membrane is known.

In an embodiment, the invention relates to a kit for determining the degree and distribution of pectin HG methyl-esterification. The kit of the invention comprises a sheet comprising a Control Zone with at least one immobilized unlabeled capture reagent and a Test Zone with at least one immobilized unlabeled anti-pectin capture antibody; at least one labeled anti-pectin detection antibody; and a Template Guide. The sheet optionally comprises a conjugate portion or comprises a sample portion and a conjugate portion. The Test Zone may comprise one, two, three, or any number of unlabeled anti-pectin HG capture antibodies that will allow the user to determine the degree and distribution of pectin HG methyl-esterification in a sample. The Control Zone may comprise one, two, three, or any number of unlabeled capture reagents that will recognize at least one labeled anti-pectin HG detection antibody used. The system may optionally comprise a container with the at least one labeled anti-pectin detection antibody. The at least one labeled anti-pectin HG detection antibody may optionally be on a conjugate pad.

In an embodiment, the invention relates to a method for determining pectin's structural/functional properties using a system of the invention. The method comprises contacting at least one labeled anti-pectin detection antibody with a pectin sample, wherein a portion of the pectin forms a pectin/labeled anti-pectin antibody complex; allowing the pectin/labeled anti-pectin antibody complex to flow through the sheet to reach the at least one immobilized unlabeled anti-pectin capture antibody at Test Zone, and allowing unbound pectin to reach the at least one immobilized unlabeled capture reagent at the Control Zone to form a color pattern; and comparing the color pattern on the sheet with a Template Guide color pattern to determine the pectin sample's homogalacturonan (HG) methyl-esterification.

In some embodiments of the invention, the sheet comprises a conjugate portion with a labeled anti-pectin detection antibody, and the method comprises contacting a pectin sample with the at least one labeled anti-pectin detection antibody on the conjugate portion. In some embodiments of the invention, the system comprises a sample portion contacting a conjugate portion comprising at least one labeled anti-pectin detection antibody, and the method comprises contacting the sample portion with a pectin sample.

Appearance of color at the Control Zone indicates that the system is functioning properly. Appearance of color at the Test Zone indicates binding of pectin to the at least one labeled anti-pectin detection antibody and to the at least one unlabeled anti-pectin capture antibody.

Unless otherwise explained, all technical and scientific terms used herein have the same meaning as commonly understood by one of ordinary skill in the art to which this disclosure belongs.

The singular terms “a”, “an”, and “the” include plural referents unless context clearly indicates otherwise. Similarly, the word “or” is intended to include “and” unless the context clearly indicate otherwise.

As used herein, the term “about” is defined as plus or minus ten percent of a recited value. For example, about 1.0 g means 0.9 g to 1.1 g.

As used herein, the terms “sheet” and “strip” are used interchangeably and refer to a narrow piece of material where reagents are immobilized, and/or through which reagents may mobilize by capillary action.

As used herein, the term “unlabeled capture antibody” refers to an unlabeled pectin-specific antibody immobilized on the sheet. In the systems of the invention, the unlabeled capture antibody may bind a pectin which is bound to a labeled detection antibody.

As used herein, the term “Control Zone” refers to a region of the sheet of the invention where at least one capture reagent is immobilized. Binding of the labeled detection antibody to a capture reagent indicates that the labeled detection antibody has moved through the sheet by capillary action.

As used herein, the terms “capture reagent,” “secondary antibody,” and “control antibody” are used interchangeably and refer to an antibody or other capture molecule immobilized in the Control Zone of a sheet of the invention.

As used herein, the term “Test Zone” refers to a region in the sheet where at least one unlabeled capture antibody has been immobilized.

As used herein, the term “Template Guide” refers to a pattern, either electronic or hard copy, that serves as a guide to interpret the results of the system or kit of the invention.

As used herein, the term “random” refers to the random distribution of methyl-esterification found in some pectins.

As used herein, the terms “pseudo-random” and “semi-random” are used interchangeably and refer to pectins with a semi-random distribution of methyl-esterification.

As used herein, a “capture reagent” is a component which specifically binds to antibodies of a specific class but not to antibodies of another class.

As used herein, a “non-loaded” detection antibody is a detection antibody that is not bound to a pectin.

As used herein, the term “sandwich” refers to a pectin captured between an immobilized unlabeled capture antibody and a labeled detection antibody.

As used herein, an “additive effect” refers to the action that occurs when the combined effect of two or more antibodies is equal to the sum of the effect of each of the antibodies given alone.

As used herein, the term “sample portion” refers to a part that serves as a sample supply portion that is capable of receiving and absorbing a liquid sample. The sample portion may be made of any material and in any form allowing the passage of liquid and the component to be detected. Specific examples of materials suitable for the sample portion include, but are not limited to, a glass fiber, an acrylic fiber, a hydrophilic polyethylene material, a dry paper, a paper pulp, a fabric, etc.

As used herein, the term “conjugate portion” refers to a portion of the sheet which is impregnated with at least one labeled anti-pectin detection antibody. The conjugate portion allows the at least one labeled anti-pectin detection antibody and the pectin in the sample to form a complex when the sample passes through the conjugate portion. The conjugate portion may be made of any material and in any form allowing the passage of liquid, the labeled detection antibody, and the labeled detection antibody bound to pectin in the sample. Specific examples of materials suitable for the conjugate portion include, but are not limited to, a glass fiber, an acrylic fiber, a hydrophilic polyethylene material, a dry paper, a paper pulp, a fabric, etc. The conjugate portion may by itself be disposed in contact with an antibody-immobilized membrane. Alternatively, the conjugate portion may be disposed in contact with the sample portion so as to receive the sample which has passed through the sample portion by a capillary flow and then transfer the sample by a capillary flow to another portion of the sheet in contact with the surface different from the contact surface with the sample pad. The selection of one or more parts of the sample portion and the conjugate portion and how the selected parts are disposed with respect to each other may be appropriately changed. Materials suitable for the conjugate pad may include, but are not limited to, paper, a cellulose compound, nitrocellulose, polyester, an acrylonitrile copolymer, a glass fiber, and a nonwoven fiber such as rayon.

As used herein, a “membrane portion” refers to a portion in the system comprising the Control Zone and Test Zone. The material used for the membrane portion may be a fiber matrix of cellulose derivative, a filter paper, a glass fiber, a cloth, a cotton, a polyester, an acrylonitrile, or nylon.

As used herein, the term “absorbent portion” refers to a liquid-absorbing part that absorbs the sample and fluid which has moved on and passed through the membrane portion to control the spread of the sample. The absorption portion may be made of, for example, filter paper.

Embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

### EXAMPLES

Having now generally described this invention, the same will be better understood by reference to certain specific examples, which are included herein only to further illustrate the invention and are not intended to limit the scope of the invention as defined by the claims.

### Example 1

### Materials

The following materials and methods were utilized in the development of a system for the assessment of pectin structural/functional properties.

Anti-pectin HG monoclonal antibodies LM19 (Rat IgM), LM20 (Rat IgM), JIM5 (Rat IgG), and JIM7 (Rat IgA) were purchased from KERAFAST (Boston, Massachusetts, USA). Goat Anti-Rat IgG and Goat Anti-Rat IgM secondary antibodies were from Jackson IMMUNORESEARCH (West Grove, Pennsylvania, USA). Goat Anti-Rat IgA secondary antibody was from BIO-RAD (Hercules, California, USA). IgM and IgG Purification Kits were purchased from ABS Advanced BIOREAGENTS (Hayward, California, USA). Protein L-Agarose (for IgA antibody purification) was from INVIVOGEN (San Diego, California, USA). The monoclonal antibodies LM19, LM20, JIM5 and JIM7 were purified by affinity chromatography following the manufacturers' instructions.

A wide variety of nitrocellulose (NC) membranes with different pore sizes and wicking rates were tested, including: lower protein binding Type CNPF-SN12-L2-H50, 8 μm (wicking rate 170 seconds per 4 cm), and CNPF-SN12-L2-H50, 10 μm (wicking rate 125 seconds per 4 cm), higher protein binding Type CNPC-SS12-L2-H50, 12 μm (wicking rate 120 seconds per 4 cm), and CN PC-SS12-L2-H50, 15 μm (wicking rate 100 seconds per 4 cm), highest protein binding Type 200CNPH-N-SS60-L2-H50 (wicking rate 200 seconds per 4 cm), 150CNPH-N-SS40-L2-H50 (wicking rate 150 seconds per 4 cm), 90CNPH-N-SS40-L2-H50 (wicking rate 90 seconds per 4), and 70CNPH-N-SS40-L2-H50 (wicking rate 70 seconds per 4 cm) purchased from MDI Membrane Technologies (Camp Hill, Pennsylvania, USA); and NC membranes FF80HP, FF120HP, FF170HP, FF80HP Plus, FF120HP Plus, and FF170HP Plus purchased from GE Healthcare (Chicago, Illinois, USA). The best results (highest sensitivity with the lowest background) were consistently achieved when using 70CNPH-N-SS40-L2-H50 (wicking rate 70 seconds per 4 cm) NC membrane with Absorbent Pad AP-080, both of which were obtained from MDI Membrane Technologies.

Purchased from CYTODIAGNOSTICS (Burlington, Ontario, Canada) were 40 nm and 80 nm NETS-Activated Gold Nanoparticles, and 80 nm and 100 nm NETS-Activated Gold NanoUrchins. GNPs were conjugated with purified anti-pectin HG monoclonal antibodies following the manufacturers' instructions.

Highly methyl-esterified citrus pectin (DM=94%), Polygalacturonic Acid (PGA), and fungal pectin methyl-esterase (PME) from Aspergillus aculeatus were purchased from Sigma (St. Louis, Missouri, USA). Plant source PME was obtained by expression of a jelly fig PME gene in yeast Pichia pastoris via expression vector pGAPZR-PME (Peng C. C., et al., 2005, “Functional expression in Pichia pastoris of an acidic pectin methylesterase from jelly fig (Ficus awkeotsang)”, J Agric. Food Chem. 53: 5612-5616; Kim Y., et al., 2014, “Characterization of charged functional domains introduced into a modified pectic homogalacturonan by an acidic plant pectin methylesterase (Ficus awkeotsang Makino) and modeling of enzyme mode of action,” Food Hydrocoll. 39: 319-329). A series of pectins with varying degrees and patterns of methyl-esterification were generated by partial de-esterification of the DM 94% citrus pectin with treatments by the plant PME (P-series), the fungal PME (F-series), or sodium hydroxide (S-series). Plant PME de-esterify pectin in a block-wise fashion, while the fungal PME de-esterify pectin more randomly, and sodium hydroxide saponification de-esterify pectin completely randomly (Ngouémazong D. E., et al., 2012, “Stiffness of Ca2+-pectin gels: combined effects of degree and pattern of methylesterification for various Ca2+ concentrations,” Carbohyd. Res. 348: 69-76; Cameron R. G., et al., 2015, “Pectin homogalacturonans: Nanostructural characterization of methylesterified domains,” Food Hydrocoll. 47: 184-190). Commercial pectins: GENU pectin type LM 12 CG-Z (DM 35%), GENU Explorer pectin 60 CS (DM 38%), GENU pectin type YM-115-L (DM 72%) and SLENDID specialty pectin type 200 (HM) were kindly provided by CP Kelco Company (Lille Skensved, Denmark).

Bovine serum albumin (BSA), polyethylene glycol 8000 (PEG 8000), and TWEEN 20 polysorbate-type non-ionic surfactant were purchased from Sigma.

### Example 2

### Pectin Assay System

The inventors assembled an illustrative system useful in the assessment of pectin structural/functional properties.

Unlabeled anti-pectin HG antibody/antibodies (capture antibody/antibodies, 0.5 mg/mL) and secondary antibody/antibodies (1.0 mg/mL) were loaded onto NC membrane (attached on an adhesive backing card) by an adjustable 0.1-2.5 μL pipette, 0.3 μL/dot. The NC membrane was dried at 37° C. for 10-15 minutes and then blocked with 3% BSA, 0.05% TWEEN 20 polysorbate-type non-ionic surfactant and 0.25% PEG 8000 (in 1×PBS, pH 7.4) and dried at 37° C. for 1 hour. The absorbent pad was attached to the adhesive backing card with an overlap of 2 mm absorbent pad covering NC membrane. The assembled card was cut into individual test strips (6 mm/strip), which were ready for immediate use or were stored in a desiccator for later use. GNP-labeled anti-pectin HG antibody/antibodies (detection antibody, OD=20) could be used immediately or aliquoted into small vials and lyophilized for later use.

The illustrative system includes everything needed to perform a test to assess pectin's structural/functional properties. As shown in FIG. 1, a system 120 of the invention may comprise at least one labeled detection antibody 122 which may optionally be in a container 124, a sheet 126 comprising an absorbent portion 128 and a membrane portion 130. The membrane portion 130 contains a Control Zone 132 with at least one unlabeled secondary antibody, and a Test Zone 134 with at least one unlabeled capture antibody. The system also requires a Template Guide, an example of which is depicted in FIG. 14. As shown in FIG. 2, a system of the invention may comprise a sheet 226 comprising an absorbent portion 228, a membrane portion 230, and a conjugate portion 236. The membrane portion 230 contains a Control Zone 232 with at least one unlabeled secondary antibody, a Test Zone 234 with at least one unlabeled capture antibody, and the conjugate portion 236 may comprise at least one labeled detection antibody 222. The system also requires a Template Guide, an example of which is depicted in FIG. 14. As shown in FIG. 3, a system of the invention may comprise a sheet 326 comprising an absorbent portion 328, a membrane portion 330, a conjugate portion 336, and a sample portion 338. The membrane portion 330 contains a Control Zone 332 with at least one unlabeled secondary antibody, a Test Zone 334 with at least one unlabeled capture antibody, and the conjugate portion 336 may comprise at least one labeled detection antibody 322. The system also requires a Template Guide, an example of which is depicted in FIG. 14.

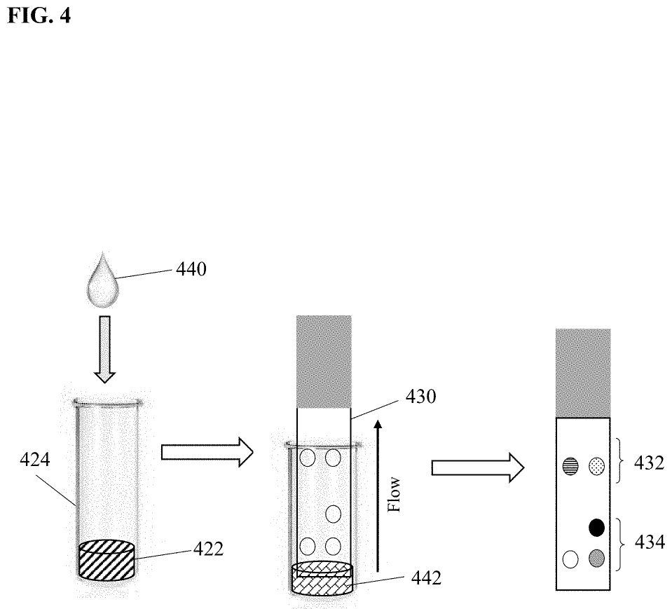

FIG. 4 depicts a schematic diagram of an illustrative method for using a system of the invention for which a schematic diagram is presented in FIG. 1 for assessing pectin's structural/functional properties. A drop (about 20 μL) of a pectin sample 440 is contacted with the at least one labeled detection antibody 422, which may be in optional container 424 to form a sample/antibody mix 442. The membrane portion 430 of the sheet is contacted with the sample/antibody mix 442. After about a five (5) minute wait a color pattern is visible on the membrane. Comparison of the color pattern obtained on the sheet with the color pattern on a Template Guide allows for the determination of the structural/functional properties of the pectin in the sample.

After a pectin sample was mixed with at least one labeled pectin-specific detection antibody, the pectin bound to the at least one labeled pectin-specific detection antibody, forming at least one pectin-detection antibody complex. The at least one pectin-detection antibody complex flowed through the membrane under capillary action until it was captured by a capture antibody through binding of the pectin to the unlabeled capture antibody. In this manner, the pectin was sandwiched between the labeled detection antibody and the unlabeled capture antibody, resulting in at least one visible color at the Test Zone. Any unbound labeled detection antibody continued to travel through the sheet until it was captured by a secondary antibody fixed at the Control Zone. Appearance of colors at the Control Zone ensures that the system is functioning properly. Buffer or excess reagent wicks to the absorbent portion. The color intensity at Test Zone can be visually inspected or can be measured with an optical strip reader.

The user-friendly system described herein is much faster and simpler than conventional immune methods such as enzyme linked immunosorbent assay (ELISA) and immuno-dot assay (IDA), which usually take at least about eight hours to perform and require well-trained laboratory operators.

### Example 3

### Sensitivity of the Assay System

The detection limit of the pectin assay system was determined to be 500 times more sensitive than that of Immuno-dot assay (IDA).

To determine the detection limit of the pectin assay system of the invention, NC membranes were prepared with an anti-IgM secondary antibody in the Control Zone and unlabeled LM19 antibody in the Test Zone. Serial dilutions of un-esterified pectin (20 μL) were added into vials containing labeled LM19 antibody, then the test strips were separately inserted into the vails. The sample/labeled antibody flowed through the Test Zone and the Control Zone via capillary action. The different pectin dilutions were 10 μg/mL, 5 μg/mL, 2.5 μg/mL, 1.25 μg/mL, 0.63 μg/mL, 0.31 μg/mL, 0.16 μg/mL, 0.08 μg/mL, 0.04 μg/mL, 0.02 μg/mL, 0.01 μg/mL, and 0.005 μg/mL. The Control Zone contained anti-rat IgM secondary antibody.

After allowing the pectin samples with the labeled antibody to flow through the NC sheets, color appeared at the Test Zone and at the Control Zone. Appearance of color at the Control Zone indicated that detection antibody had been labeled successfully and the pads and NC membrane were working properly; appearance of color at the Test Zones indicated that at that pectin concentration, pectin in a sample was detectable by the system. FIG. 5 shows photographs of the results obtained. Not wishing to be bound by theory, it is believed that the signal is darker at the Test Zone and lighter at the Control Zone when testing 10 μg/mL pectin, indicating that most of the labeled detection antibody was bound to pectin. And the signal is lighter at the Test Zone and darker at the Control Zone when testing 0.02 μg/mL pectin, indicating that most of the labeled detection antibody was not bound to pectin. AT 0.02 μg/mL or a higher concentration, the signal at the Test Zone was visible; while at 0.01 μg/mL or 0.005 μg/mL, the signal at the Test Zone was barely or not visible by eyes. The results indicate the detection limit of the assay taught herein is about 0.02 μg/mL.

Color intensity at the Test Zones of the strips was quantified by ImageJ software, and a standard curve was generated based on the color intensity and is shown in FIG. 6. The data shown in the graph represents Mean±SEM of 3 independent experiments. This graph indicates that there is a good correlation (R²=0.99) between the concentration of the pectin tested and the color intensity in the Test Zone, and the detection limit of the assay taught herein is between 0.01 μg/mL and 0.02 μg/mL. This detection limit is 500 times lower than that of IDA, which is 10 μg/mL (Christiaens S., et al., 2011, “Anti-homogalacturonan antibodies: A way to explore the effect of processing on pectin in fruits and vegetables?” Food Res. Int. 44: 225-234). Not wanting to be bound by theory, it is believed that the reason why the method taught here has such high sensitivity is because the detection signal is concentrated and amplified due to pure antibody being concentrated in a small area (Test Zone), plus the amplification effect of binding with a second antibody. This results in a dramatically increased sensitivity and a much lower detection limit compared to conventional immunoassays. The lower detection limit makes the method taught herein applicable to a wider range of samples including those with low pectin content.

The results obtained in this Example show that the detection limit of the pectin assay system of the invention is about 500 times more sensitive than that of IDA.

### Example 4

### Selectivity of Antibodies

The pectin structural property-discriminating abilities of antibodies LM19, LM20, JIM5 and JIM7 in ICA were evaluated with model pectins 0, 94; random distribution pectins S28, S53, S72, and S81; semi-random distribution pectins F30, F50, F60, F70, and F80; and block-wise distribution pectins P30, P47, and P70. Appearance of color at the Test Zone indicated a positive result. The color intensity of the results was quantified with ImageJ software.

Different from a regular ICA, which is aimed at detecting a target in a sample, the goal of the ICA run in the instant application was to discern pectin structural properties that are related to its functionality. Pectin HG binding antibodies such as LM19, LM20, JIM5 and JIM7 have been evaluated in an IDA regarding their ability to discern different structural features of pectin. In the IDA, the “resolution” of these antibodies was limited to either totally un-esterified (PGA) or fully-esterified (DM>90) pectin (Christiaens S., et al., supra); for partially esterified pectins, the degree and pattern of methyl-esterification were indistinguishable. As seen in FIG. 7A to FIG. 7D, in a sandwich ICA of the invention, the resolution of these antibodies improved to varying degrees. In the assay taught herein, a positive result requires that a pectin molecule bind to two antibody molecules (a labeled detection antibody and an unlabeled capture antibody) at the same time; while in an IDA, a positive result only requires a pectin molecule bind to one antibody molecule. FIG. 7A to FIG. 7D show the results of evaluating the abilities of LM19, LM20, JIM5 and JIM7 antibodies to discriminate different structural types of pectin in Sandwich ICA by testing a series of model pectins. Each of the antibodies was labeled by GNP as detection antibody and was immobilized on the Test Zone as unlabeled capture antibody; and their secondary antibodies were immobilized separately on the Control Zone. After adding a model pectin sample, a positive signal (color) appeared on the Test Zone is an indication of the formation of a “sandwich” where pectin is captured between an immobilized unlabeled antibody and a labeled antibody. The results obtained when using LM19 antibody are shown in FIG. 7A. This figure shows positive results for un-esterified pectin (0), partially de-esterified pectins with a semi-random distribution (F30, F50, F60, F70, and F80) or ordered, block-wise distribution (P30, P47, and P70) of methyl-esterification. And negative results for fully-esterified pectin (DM>90%) or partially de-esterified pectins with a random distribution of methyl-esterification (S28, S53, S72, and S81). The results obtained when using LM20 antibody are shown in FIG. 7B. This figure shows positive results for fully or partially esterified pectins, but the signals were weak for those partially esterified pectins with a semi-random distribution of methyl esterification. The LM20 antibody did not generate a positive result for un-esterified (0) pectin. The results obtained when using JIM5 antibody are shown in FIG. 7C. This figure shows that the results of JIM5 were similar to that of the LM19, but the signals were much weaker. The results obtained when using JIM7 antibody are shown in FIG. 7D. This figure shows positive results for fully or partially esterified pectins except the LM (DM<50%) pectin with a block-wise distribution of methyl esterification. And a negative result for un-esterified pectin (0). The color intensity in FIG. 7A to FIG. 7D was quantified using ImageJ software and the results are shown in FIG. 8A to FIG. 8D.

The results obtained in this Example indicate that the assessment of the structural features of pectin cannot depend on only one ICA probing by a single antibody pair. Therefore, a multiplex detection format of ICA was designed.

### Example 5

### Multiplex Ica Test Strip with Single Detection Antibody

A multiplex ICA test strip was developed and assayed using a GNP-labeled detection antibody (LM19, LM20. JIM5, or JIM7).

To formulate a membrane portion to be used in a system of the invention capable of discriminating between different pectin functional forms, all four HG antibodies were included as capture antibodies on a single membrane portion (a schematic of which is depicted in FIG. 9), working independently with each of the four GNP-labeled antibodies. Model pectins S28 and S72 of random distribution, F30 and F70 of semi-random distribution, and P30 and P70 of block-wise distribution were tested by using a test strip with a membrane portion as depicted in FIG. 9, and a labeled detection antibody (LM19, LM20, JIM5, or JIM7). The results are shown in FIG. 10A to FIG. 10D.

These results indicated that the multiplex ICA test strip used with detection antibodies LM19 (FIG. 10A), LM20 (FIG. 10B), or JIM7 (FIG. 10D) clearly discriminated the pectins with a block-wise distribution of methyl-esterification from those with a random or semi-random distribution; while JIM5 (FIG. 10C) did not clearly discriminate between pectins. The LM20 (FIG. 10B) and the JIM7 (FIG. 10D) detection antibodies also discriminated between LM and HM pectins for all the three patterns of methyl-esterification, with an additional positive signal on the site of the JIM7 capture antibody for HM pectins. The signal patterns generated in the Test Zone when using the LM20 detection antibody (FIG. 10B) were unique for each of the pectin types. However, due to the fact that there was no positive signal at any of the four capture antibody sites for the semi-randomly distributed LM (F30) pectin when using the LM20 detection antibody, it was not possible to tell if a semi-randomly distributed low degree (<50%) of methyl-esterification pectin was present in the sample or if there was no pectin present in the tested sample. As seen in FIG. 10D, although there was at least one positive signal in the test zone for each of the pectin types when using the JIM7 detection antibody, the signal pattern for randomly and semi-randomly distributed LMs (S28 and F30) was not distinguishable.

The results obtained in this Example indicate that a multiplex format with multiple capture antibodies immobilized in the Test Zone would greatly improve the capability of an ICA test strip to discern different pectin functional forms, especially when using labeled LM20 or JIM7 as detection antibody.

### Example 6

### Multiplex Ica Test Strip with Two Detection Antibodies

A multiplex ICA system capable of discriminating between different pectin functional forms was developed. The system includes three capture antibodies immobilized on the Test Zone and two detection antibodies labeled with different colors of Gold NanoParticies. The capability of the system was validated by testing model pectins and commercial pectins.

In the multiplex ICA of Example 5, JIM7 detection antibody generated at least one positive signal in the test zone for each of the pectin types, but the signal patterns were not specific; LM20 detection antibody generated specific signal patterns, but the signal for one of the pectin types was negative. Both the JIM7 and the LM20 detection antibodies discriminated between LM and HM, but the signal obtained was weak. Therefore, a system was designed, which includes an 80 nm spherical GNP (red)-labeled JIM7 and an 80 nm star-shaped GNP (blue)-labeled LM20 as detection antibodies, and a membrane portion with unlabeled anti-IgM and anti-IgA on the Control Zone, and unlabeled capture antibodies LM19, LM20 and JIM7 on the Test Zone. The membrane portion of the system was schematically diagrammed in FIG. 11. The ability of the system to discern different structural/functional forms of pectin was evaluated using model pectins and commercial pectins.

Model pectins with random distribution tested were S28 (LM) and S72 (HM); with semi-random distribution tested were F30 (LM) and F70 (HM); with block-wise distribution tested were P30 (LM) and P70 (HM). The results obtained when using a membrane portion as diagrammed in FIG. 11, with a JIM7 antibody labeled with 80 nm spherical GNPs (red), and an LM20 antibody labeled with 80 nm star-shaped GNPs (blue) are shown in FIG. 12. This figure shows that a specific pattern was obtained with each of the model pectins tested, indicating the developed ICA system is capable of discriminating between different structural/functional types of pectin.

As seen in FIG. 12, when testing model pectins with known structural/functional properties, unique patterns or colors of positive signals were generated in the Test Zone for each of the pectin HG structural types that are related to pectin's functionality. The pectins with block-wise distribution of methyl-esterification, which have the highest quality as gelling agents or stabilizers in foods, were also the most distinguishable types of pectins in the test using the system of the invention by showing clear positive signals at both the LM19 and the LM20 unlabeled capture antibody sites. A positive signal at the JIM7 capture antibody site allowed for the identification of an HM or LM pectin, which informs whether a pectin can be used for low sugar or high sugar content foods. The pectins with random or semi-random distribution of methyl ester units were discriminated by the position (at either the LM19 or the LM20 site) and the color (purple or red) of the positive signal on the bottom line of the Test Zone.

Commercial pectins used were GENU pectin type LM 12 CG-Z, DM 35%; GENU Explorer pectin 60 CS, DM 38%; GENU pectin type YM-115-L, DM 72%; SLENDID specialty pectin type 200, which is labeled as an HM pectin, but for which its DM was not provided. These four commercial pectins were tested using the system with a membrane portion as depicted in FIG. 11 with a JIM7 detection antibody labeled with 80 nm spherical GNPs (red), and an LM20 detection antibody labeled with 80 nm star-shaped GNPs (blue). The results are presented in FIG. 13, where the pectins tested are indicated at the top of the figure. Commercial-1 is GENU pectin type LM 12 CG-Z, DM 35% (LM); Commercial-2 is GENU Explorer pectin 60 CS, DM 38% (LM); Commercial-3 is GENU pectin type YM-115-L, DM 72% (HM); and Commercial-4 is SLENDID specialty pectin type 200, HM.

The results shown in FIG. 13 indicate that these commercial pectin products have block-wise distribution of methyl-esterification, which is a desirable trait for a gelling agent or a stabilizer to be used in foods. The results also show that, using a system of the invention, the HM pectins were clearly distinguishable from the LM pectins, and that these results were consistent with the information provided by the product provider. These results indicate that it is possible to determine commercial pectins' HG methyl-esterification by using the system of the invention with a membrane portion as depicted in FIG. 11, a JIM7 detection antibody labeled with 80 nm spherical GNPs, and a LM20 detection antibody labeled with 80 nm star-shaped GNPs.

Based on the experimental data obtained, a judgment criteria for pectin structural properties tested using a multiplex ICA of the invention with a membrane portion as diagramed in FIG. 11, with a JIM7 antibody labeled with 80 nm spherical GNPs and a LM20 antibody labeled with 80 nm star-shaped GNPs may be summarized as in the Template Guide shown in FIG. 14. Appearance of one blue dot (shown filled in gray) and one red dot (shown with vertical stripes) on the Control Zone indicates that the system is functioning properly. The results are judged based on the pattern/color of the dots appearing on the Test Zone. A developed test strip with only one purple dot (shown with upward diagonal stripes) on the immobilized LM19 at the Test Zone indicates a pectin with LM random distribution. A developed test strip with two purple dots (shown with vertical stripes), one on the immobilized JIM7 and one on the immobilized LM20 at the Test Zone indicates a pectin with HM random distribution. A developed test strip with only one red dot (shown with vertical stripes) on the immobilized LM19 at the Test Zone indicates a pectin with LM semi-random distribution. A developed test strip with one red dot (shown with vertical stripes) on the immobilized LM19 and one purple dot (shown with upward diagonal stripes) on the immobilized JIM7 at the Test Zone indicates a pectin with HM semi-random distribution. A developed test strip with one dark purple dot (shown with downward diagonal stripes) on the immobilized LM19 and a lighter purple dot (shown with upward diagonal stripes) on the immobilized LM20 at the Test Zone indicates a pectin with LM block-wise distribution. A developed test strip with one dark purple dot (shown with downward diagonal stripes) on the immobilized LM19 and two lighter purple dots (shown with upward diagonal stripes), one on the immobilized LM20 and one on the immobilized JIM7 at the Test Zone, indicates a pectin with HM block-wise distribution.

We claim:

1. A system for determining degree and distribution of pectin homogalacturonan (HG) methyl-esterification, the system comprising:
a sheet comprising a Control Zone with at least one immobilized unlabeled capture reagent and a Test Zone with at least one immobilized unlabeled anti-pectin capture antibody;
at least one labeled anti-pectin detection antibody; and
a Template Guide;
wherein the sheet optionally comprises a container, or a conjugate portion, or a sample portion and a conjugate portion.

2. The system of claim 1, wherein the system comprises a container, and the at least one labeled anti-pectin detection antibody is in the container.

3. The system of claim 1, wherein the sheet comprises a conjugate portion, and the at least one labeled anti-pectin detection antibody is on the conjugate portion.

4. The system of claim 1, wherein the at least one labeled anti-pectin detection antibody is JIM7, or LM20.

5. The system of claim 1, wherein the at least one immobilized unlabeled anti-pectin capture antibody is JIM7, LM19, or LM20.

6. The system of claim 1, wherein the Control Zone comprises at least two unlabeled capture reagents.

7. The system of claim 1, wherein the Test Zone comprises at least three unlabeled anti-pectin capture antibodies.

8. The system of claim 7, wherein the Test Zone comprises JIM7, LM19, and LM20 antibodies.

9. The system of claim 1, wherein the system comprises at least two labeled anti-pectin detection antibodies.

10. The system of claim 9, wherein the at least two labeled anti-pectin detection antibodies are JIM7 and LM20.

11. A method for determining pectin's structural/functional properties using a system of claim 1, the method comprising:
contacting at least one labeled anti-pectin detection antibody with a pectin sample, wherein a portion of the pectin forms a pectin/labeled anti-pectin antibody complex;
allowing the pectin/labeled anti-pectin antibody complex to flow through the sheet to reach the at least one immobilized unlabeled anti-pectin capture antibody at Test Zone, and allowing unbound pectin to reach the at least one immobilized unlabeled capture reagent at the Control Zone to form a color pattern; and
comparing the color pattern on the sheet with a Template Guide color pattern to determine the pectin sample's homogalacturonan (HG) methyl-esterification.

12. The method of claim 11, wherein the at least one labeled anti-pectin detection antibody is in a container, and the pectin sample is added to the container to contact the at least one labeled anti-pectin detection antibody.

13. The method of claim 11, wherein the sheet comprises a conjugate portion with the at least one labeled anti-pectin detection antibody, and the pectin sample is added to the conjugate portion to contact the at least one labeled anti-pectin detection antibody.

14. The method of claim 11, wherein the sheet comprises a sample portion and a conjugate portion with the at least one labeled anti-pectin detection antibody, and the pectin sample is added to the sample portion and allowed to flow through the sample portion to the conjugate portion to contact the at least one labeled anti-pectin detection antibody.

15. The method of claim 11, wherein the Control Zone comprises at least two unlabeled capture reagents.

16. The method of claim 11, wherein the Test Zone comprises at least three unlabeled anti-pectin capture antibodies.

17. The system of claim 16, wherein the Test Zone comprises JIM7, LM19, and LM20 antibodies.

18. The system of claim 11, wherein the system comprises at least two labeled anti-pectin detection antibodies.

19. The system of claim 18, wherein the at least two labeled anti-pectin detection antibodies are JIM7 and LM20.
